(19) United States
(12) Patent Application Publication
(10) Pub. No.: US 2016/0278664 A1
(43) Pub. Date: Sep. 29, 2016

(54) FACILITATING DYNAMIC AND SEAMLESS BREATH TESTING USING USER-CONTROLLED PERSONAL COMPUTING DEVICES

(71) Applicant: INTEL CORPORATION, Santa Clara, CA (US)

(72) Inventors: MONDIRA D. PANT, Westborough, MA (US); MEGGIE HAKIM, Feldkirchen (DE); YOUSSRY BOTROS, Aliso Viejo, CA (US); TANAY KARNIK, Portland, OR (US)

(73) Assignee: INTEL CORPORATION, Santa Clara, CA (US)

(21) Appl. No.: 14/671,069

(22) Filed: Mar. 27, 2015

(51) Int. Cl. A61B 5/08; A61B 5/00

(52) U.S. Cl. CPC A61B 5/082; A61B 5/746; A61B 5/742; A61B 5/7455; A61B 5/7221; A61B 5/0022

(57) ABSTRACT

A mechanism is described for facilitate dynamic and seamless breath testing at computing devices according to one embodiment. A method of embodiments, as described herein, includes detecting air exhaled by a user into a first computing device, where the air includes breath associated with the user. The method may further include sensing the breath in the air, obtaining a sample of the breath, and evaluating the sample, and generating a message based on the evaluation of the sample. The method may further include presenting, via one or more output components, the message to the user via a user interface, where the message includes results of the evaluation of the breath sample.

### FIELD

Embodiments described herein generally relate to computers. More particularly, embodiments relate to facilitating dynamic and seamless breath testing using user-controlled personal computing devices.

### BACKGROUND

Conventional breath sensing techniques are rather cumbersome and not user-friendly as they require special equipment along with additional consumables, such as nose clips and mouthpieces, and lack in performing intelligent breath analysis. Given the unfriendly nature and limited use of such conventional techniques, most users choose to shy away from them which can often lead to various diseases (e.g., diabetes) going undetected.

### BRIEF DESCRIPTION OF THE DRAWINGS

Embodiments are illustrated by way of example, and not by way of limitation, in the figures of the accompanying drawings in which like reference numerals refer to similar elements.

FIG. 1 illustrates a computing device employing a dynamic breath testing mechanism according to one embodiment.

FIG. 2A illustrates a dynamic breath testing mechanism according to one embodiment.

FIG. 2B illustrates an architectural placement of a selective set of components of a dynamic breath testing mechanism according to one embodiment.

FIG. 2C illustrates a personal device according to one embodiment.

FIG. 3A illustrates a graph showing a normal capnography for exhaled breath.

FIG. 3B illustrates graphs showing humidity monitoring during breathing based on exhaled humidity.

FIG. 4 illustrates computer system suitable for implementing embodiments of the present disclosure according to one embodiment.

FIG. 5 illustrates computer environment suitable for implementing embodiments of the present disclosure according to one embodiment.

FIG. 6A it illustrates a method for performing breath testing tasks according to one embodiment.

FIG. 6B illustrates a method for monitoring and evaluation of personal devices and users in relation to breath testing according to one embodiment.

### DETAILED DESCRIPTION

In the following description, numerous specific details are set forth. However, embodiments, as described herein, may be practiced without these specific details. In other instances, well-known circuits, structures and techniques have not been shown in details in order not to obscure the understanding of this description.

Embodiments offer breath sensing and analysis on smart personal devices that are seamless and intuitive to use, where smart personal devices may include smart mobile devices, such as toothbrushes, smartphones, bracelets, watches, glasses, etc. Embodiments provide for safe, non-invasive, and convenient breath sampling and analysis on smart personal devices to offer 1) monitoring of health-related compounds, such as acetone for diabetes, nitric oxide for asthma, etc.; 2) clinical diagnostics based on breath odors; 3) detection of breath compounds, such as ethanol for alcohol monitoring, hydrogen disulfide for halitosis for increased wellbeing; 4) monitoring ambient air compounds for air monitoring application, etc.

In additional to offering breath sensing and analysis on a user's smart personal device, embodiments further provide for communication capabilities where the smart personal device can stay in communication with a central computing system to facilitate first responders and other healthcare professionals (e.g., doctors, nurses, etc.) to have the ability to continuously monitor the user's health and warn the user in case of an emergency, etc. These communication capabilities provide for integrated personal devices to offer: 1) continuous real-time monitoring for providing early detection: 2) portability and miniature nature of the system for mobile device integration; 3) low-powered system running on batteries; 4) compatibility with CMOS processing for smartphone integration; 5) module systems that are customized to detect a variety of compounds with no hardware changes, etc.

It is to be noted that various smart mobile computing devices, such as tablet computers, smartphones, toothbrushes, wearable devices (e.g., head-mounted displays, wearable glasses, watches, wristbands, clothing items, jewelry, etc.), and/or the like, may be collectively referred to as “personal computing devices”, “personal computers”, “personal devices”, or simply “devices” throughout this document. Similarly, various healthcare professionals, such as first responders, emergency room personnel, doctors, nurses, medical administrative staff, etc., may be collectively referred to as “healthcare professionals”, “health professionals”, “medical professionals”, or simply “medical staff” throughout this document. For example and in one embodiment, personal devices and medical staff may be performed in various modalities, such as visual, auditory, haptic, olfactory, etc. Similarly, employing Global Positioning System (GPS) at personal devices may allow the medical staff to stay aware of the exact locations of the personal devices and thus their corresponding users.

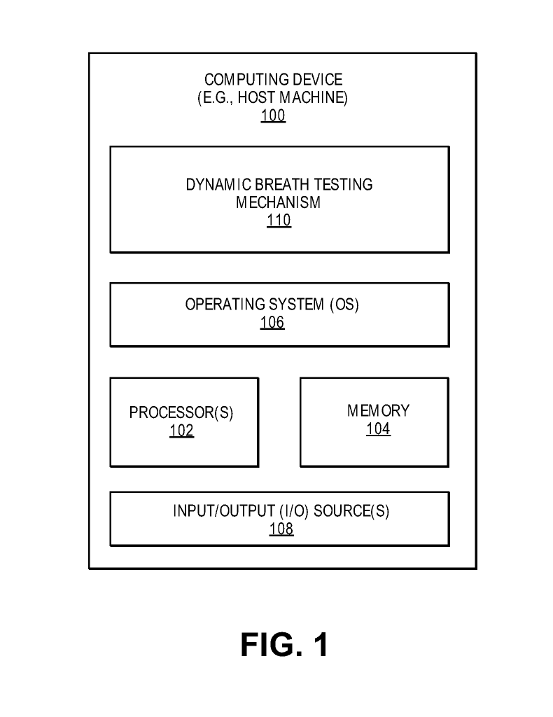

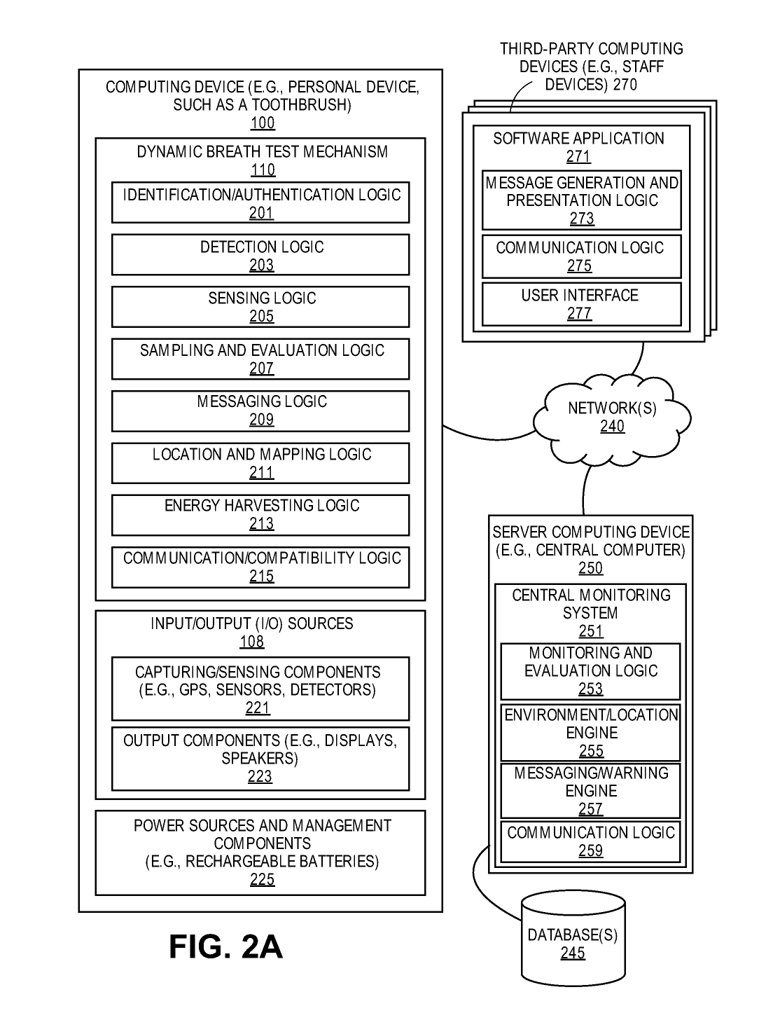

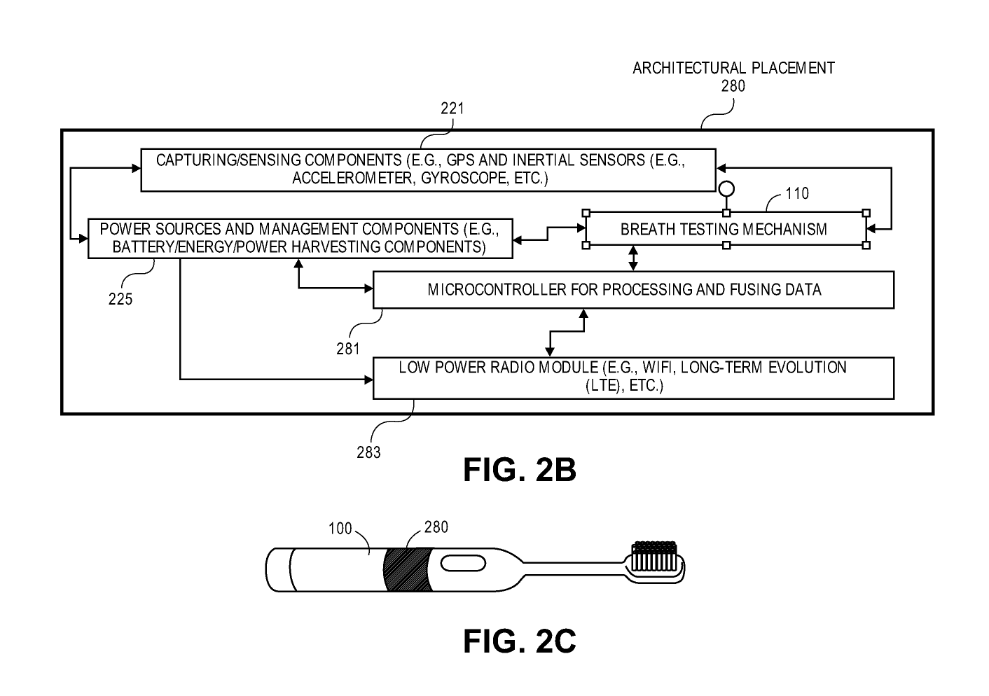

FIG. 1 illustrates a computing device 100 employing a dynamic breath testing mechanism 110 according to one embodiment. Computing device 100 serves as a host machine for hosting a personal device-based dynamic breath testing mechanism (“breath testing mechanism”) 110 that includes any number and type of components, as illustrated in FIG. 2, to efficiently employ one or more components to dynamically facilitate personal device-based and user-controlled breath sensing and analysis as will be further described throughout this document.

It is contemplated and to be noted that although “breath” is referenced throughout the document for brevity, clarity, and ease of understanding, embodiments are not limited as such as that other manners of testing, such as saliva testing, may be employed to achieve similar or the same results. For example, computer device 100 (e.g., smart toothbrush) may have a button for mode switching as facilitated by breath testing mechanism 110, wherein the mode switching button may be used by the user of computing device 100 to switch from one mode of testing to another, such as from breath to saliva and vice versa.

Computing device 100 may include any number and type of data processing devices, such as large computing systems, such as server computers, desktop computers, etc., and may further include set-top boxes (e.g., Internet-based cable television set-top boxes, etc.), global positioning system (GPS)-based devices, etc. Computing device 100 may include mobile computing devices serving as communication devices, such as cellular phones including smartphones, personal digital assistants (PDAs), tablet computers, laptop computers (e.g., Ultrabook™ system, etc.), e-readers, media internet devices (MIDs), media players, smart televisions, television platforms, intelligent devices, computing dust, media players, toothbrushes, head-mounted displays (HMDs) (e.g., wearable glasses, such as Google® Glass™ head-mounted binoculars, gaming displays, military headwear, etc.), and other wearable devices (e.g., smartwatches, bracelets, smartcards, jewelry, clothing items, etc.), and/or the like.

Computing device 100 may include an operating system (OS) 106 serving as an interface between hardware and/or physical resources of the computer device 100 and a user. Computing device 100 further includes one or more processors 102, memory devices 104, network devices, drivers, or the like, as well as input/output (I/O) sources 108, such as touchscreens, touch panels, touch pads, virtual or regular keyboards, virtual or regular mice, etc.

It is to be noted that terms like “node”, “computing node”, “server”, “server device”, “cloud computer”, “cloud server”, “cloud server computer”, “machine”, “host machine”, “device”, “computing device”, “computer”, “computing system”, and the like, may be used interchangeably throughout this document. It is to be further noted that terms like “application”, “software application”, “program”, “software program”, “package”, “software package”, “code”, “software code”, and the like, may be used interchangeably throughout this document. Also, terms like “job”, “input”, “request”, “message”, and the like, may be used interchangeably throughout this document. It is contemplated that the term “user” may refer to an individual or a group of individuals using or having access to computing device 100.

FIG. 2A illustrates a dynamic breath testing mechanism 110 according to one embodiment. In one embodiment, breath testing mechanism 110 include any number and type of components, such as (without limitation): identification/authentication logic 201; detection logic 203; sensing logic 205; sampling and evaluation logic 207; messaging logic 209; location and mapping logic 211; energy harvesting logic 213; and communication/compatibility logic 215. Computing device 100 further includes I/O sources 108 having any number and type of capturing/sensing components 221 (e.g., GPS, hardware sensors, hardware detectors, etc.), output components 223 (e.g., display devices/screen, speakers, etc.), power sources and management components 225 (e.g., rechargeable batteries, wireless charging plates, etc.).

As an initial matter, it is contemplated and to be noted that although “breath” or “breath testing” is referenced throughout this document for brevity, clarity, and ease of understanding, embodiments are not limited as such as that other forms of testing, such as saliva testing, may be employed to achieve similar or the same results. For example, computer device 100 (e.g., smart toothbrush) may have a button for mode switching as facilitated by breath testing mechanism 110, wherein the mode switching button may be pressed or switched by the user of computing device 100 to switch between modes of testing, such as from breath to saliva and vice versa.

Computing device (hereinafter, also referred to as “personal device”) 100 hosting breath testing mechanism 110 may be in communication with another computing device (hereinafter, also referred to as “server computer” or “central computer”) 250, serving as a server computer, over one or more networks, such as network 240 (e.g., Cloud network, the Internet, intranet, Internet of Things (“IoT”), proximity network, Bluetooth, etc.). Further, personal device 100 and/or central computer 250 may be in communication with one or more third-party computing devices (hereinafter, also referred to as “client devices” or “staff devices”) 270 over network 240, where staff devices 270 may include any number and type of computing devices (e.g., desktop computers, portable or mobile computers, such as smartphones, tablet computers, laptops, etc.) that are available to and accessed by members of medical staff to keep in communication with and stay aware of the condition of the user of personal device 100.

Central computer 250 may include central monitoring system 251 including one or more components, such as (without limitation): monitoring and evaluation engine 253; environment/location engine 255; message/warning engine 257; and communication engine 259. Central computer 250 may be further in communication with more repositories or databases, such as database 245, where any amount and type of data (e.g., real-time data, historical contents, metadata, resources, policies, criteria, rules and regulations, upgrades, etc.) may be stored and maintained. Similarly, staff devices 270 may include software application 271 having one or more components, such as (without limitation): message generation and presentation logic 273; communication logic 275; and user interface 277.

As aforementioned, embodiments provide for a breath sensing and analyzing technique that is offered via personal device-based hardware to enable daily, seamless, and incidental sensing and analyzing of breath without having to use any conventional equipment or additional consumables, such as nose clips, mouthpieces, etc. As previously listed, personal device 100 may include any number and type of smart mobile devise, such as smartphones, bracelets, lockets, watches, etc., and that embodiments are not limited to any particular smart device; however, for the sake of brevity, clarity, and ease of understanding, throughout this document, toothbrush is use as an example of personal device 100 employing breath testing mechanism 110.

For example, having a toothbrush as personal device 100, it is contemplated that personal device 100 is likely to be used regularly twice a day by the user, allowing breath testing mechanism 110 to detect alveolar air to enable seamless and accurate breath detection. In some embodiments, personal device 100 and/or the user of personal device 100 may be identified and authenticated by identification/authentication logic 201. For example, since a toothbrush is considered a personal device, breath testing mechanism 110 may be specifically set to be used by the user of personal device 100 such that identification/authentication logic 201 may be automatically triggered each time personal device 100 is used or breath testing mechanism 110 is turned on so that the user may be identified and only the user's breath is detected and used for analysis. In one embodiment, identification/authentication logic 201 may be option and not part of breath testing mechanism 110.

In one embodiment, various components (e.g., detection logic 203, sensing logic 205, etc.) of breath testing mechanism 110 along with other components, such as one or more hardware detectors/sensors of capturing/sensing components 221, of personal device 100 may work together to provide for an integrated system offering easy, seamless, and consistent user experiences, such as (without limitation): 1) innocuous tracking of breath; 2) consistent time of tracking of breath on a daily basis; 3) without any additional gadgets (e.g., smart phones, smart wearables, etc.); 4) untainted tracking of breath (since, in case of toothbrush being personal device 100, brushing of teeth is likely to happen first thing in the morning before the user has eaten any food); etc.

Further, for example, having a toothbrush as personal device 100, it may be applied by the user for cleaning teeth twice a day which may be used for breath detection and analysis. Further, as with today's motorized toothbrushes, there may be a turn on/off switch on personal device 100 to allow the user to control and decide on whether to turn on/off breath testing mechanism 110. In one embodiment, there may be just a single switch to allow for a period of time (e.g., 15 seconds) dedicated towards breath sensing and analysis before allowing for normal brushing to commence. Since toothbrushes are exposed to water on a regular basis, personal device 100 may be waterproof to prevent any possible damage. As aforementioned, this dynamic and seamless breath sensing and analyzing capability, as facilitated by breath testing mechanism 110, may be integrated into other smart mobile devices, such as smartphones and wearables (e.g., smartwatches, bracelets, etc.).

Referring back to the various components of breath testing mechanism 110, in one embodiment, detection logic 203 may serve as an alveolar breath detector to enable detection of alveolar air and the sensing of breath in it by sensing logic 205. In some embodiments, detection logic 203 and sensing logic 205 may be formed as a single logic performing multiple tasks. The alveolar breath may refer to the breath from the deepest part of the lungs and it is contemplated that any air exhaled by the user may include a mixture of alveolar and ambient air that is retained in the respiratory dead space. The biomarkers of interest originate from the alveolar air which has been in contact with the blood inside the alveoli. This is one of the reasons that some of the conventional breath sensing techniques require the users to go through a lunch washout by breathing pure air for a period of time (typically, 4-30 minutes) an then exhale the total lunch capacity (TLC) in a single-step process lasting around another period of time (about 3 seconds) through a consumable mouthpiece while using a nose clip to exclude nasal or gas entertainment. Some other conventional techniques may not require a lunch washout, but still require the user to breathe into a consumable mouth piece for some time, typically 2-3 seconds, which can cause a great deal of difficulty for many individuals, such as those with lunch or airway problems (e.g., pneumonia, asthma, cardiac issues, etc.).

In one embodiment, detection logic 203 may be used to detect the air that is crucial for detection of alveolar breath which may then be used for sensing and analyzing breath. For example and in one embodiment, one or more portions of the detected air may contain or represent alveolar or alveolar breaths that are then sensed by sensing logic 205 for sampling and further processing. For example, the air having at least a portion of alveolar breath having the aforementioned biomarkers of interest may be detected by detection logic 203 using one or more alveolar breath detection techniques, such as (without limitation): 1) Non-Dispersive Infra-Red (NDIR) technique for carbon dioxide (CO₂) monitoring; 2) relative humidity sensing technique, etc.

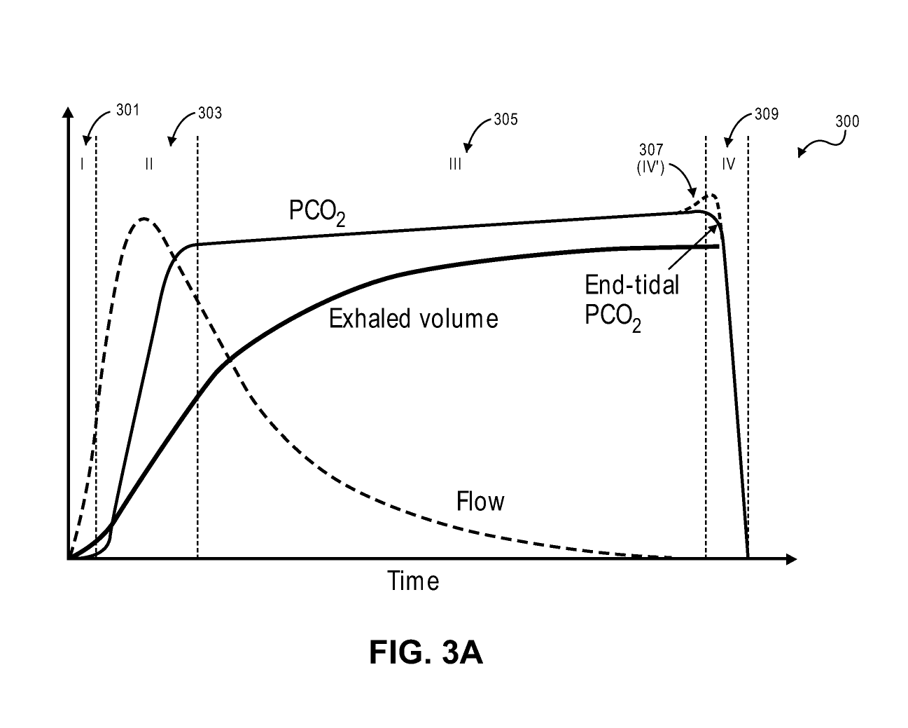

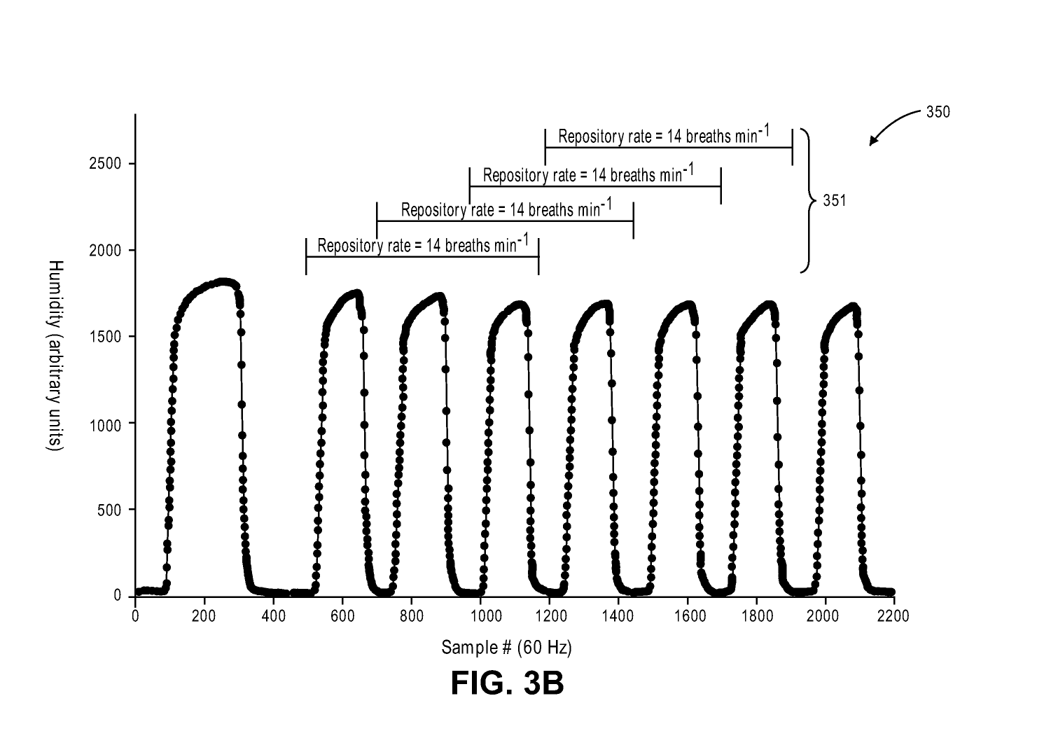

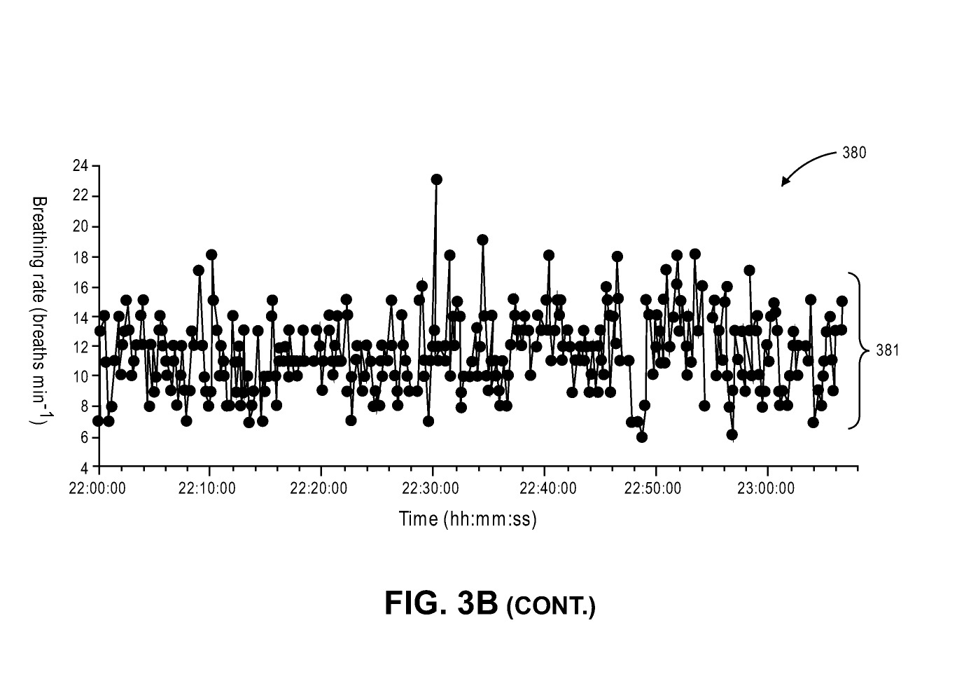

When detecting CO₂ concentration as a marker of alveolar breath, several phases may be distinguished in a capnography (e.g., a tool for monitoring concentration or partial pressure of CO₂ in the respiratory gases) as illustrated in FIG. 3A. Referring now to FIG. 3A, it illustrates a graph 300 showing a normal capnography for exhaled breath, where inspiration and the first portion of expiration is assumed during which dead space gas is exhaled as there is no CO₂ represented in phase I 301. As expiration continues, a short phase of the full capnograph is recognized and represented in phase II 303, with a rapid upstroke toward the alveolar plateau, representing the rising front of CO₂. Phase III 305, also referenced as the alveolar plateau, represents the constant or slowly up sloping part of the capnograph. Phase III 305 is followed by phase IV′ 307 which represents the beginning of an end-tidal of CO2, leading to a sharp drop in CO2 in the final illustrated phase, phase IV 309. Referring back to FIG. 2A, in one embodiment, sensing logic 205 may serve as a CO₂ sensor for the sampling of breath which may be triggered during phase III 305 of FIG. 3A to secure the sampling of, for examples, only the alveolar air. For reference, the normal values of CO₂ may be around 55-6% which may be equivalent of 35-45 mm Hg.

Similarly, in another embodiment, in compliance with the relative humidity sensing technique, sensing logic 205 may be used to sense relative humidity as a marker of alveolar breath in the detected respiratory air. For example, referring now to FIG. 3B, it illustrates graphs 350, 380 showing humidity monitoring during breathing based on exhaled humidity. In one embodiment, as illustrated in FIG. 3B, the relative humidity measured during breath is also affected from the source of the breath as illustrate with reference to respiratory rates 351 of graph 350 that are based on breathing rates 381 of graph 380. For example, referring back to FIG. 2A, the alveolar air, as detected by detecting logic 203, may include a 100% relative humidity and so monitoring the change of relative humidity during each breath exhale, as sensed by sensor logic 205, may be used as another parameter to decide when to sample the alveolar air.

In one embodiment, upon having the sensed information obtained from one of CO₂ and relative humidity techniques via sensing logic 205, sampling and evaluation logic 207 may be used to identify the source of the breath (e.g., dead space, alveolar, etc.) and sample the breath using one or more of the aforementioned techniques. For example, upon having the user decide when to take the breath test, such as by switching-on an on/off switch on personal device 100, to intentionally subject to a dedicated breath testing exercise which may then activate sampling and evaluation logic 207 to perform breath sampling, such as sample the breath when, for example, the source of breath is alveolar and indicate to the user when a sufficient amount of breath have been collected. Similarly, for example, during brushing of the teeth, where a toothbrush is personal device 100, the sensing of the breath using sensing device 205 may activate sampling and evaluation logic 207 to sample an amount of breath until the brushing is completed, such as when the source of the breath is alveolar. Once the sampling and accumulation of the breath is completed, the accumulated breath amount may then be evaluated and analyzed by sampling and evaluation logic 207.

It is contemplated that any number and type of detector and sensors, such as hardware-based detectors and sensors, may be employed as part of capturing/sensing components 221 to work in communication with various components, such as detecting logic 203 and sensing logic 205, to be used for performing various tasks relating to detection of air, sensing breath, etc. For example, personal device 100 being a toothbrush, sensing elements may be small in size to fit personal device 100, such as sensing elements having various elements and functionalities, such as chemical separation and biomarker detection, gas pumping, micro-electrical mechanism systems (MEMS), standard integrated circuit technology, such as complementary metal-oxide semiconductor (CMOS), etc.

In one embodiment, messaging logic 209 may be used in communication with central computer 250 and staff devices 270, over network 240, for messaging purposes. For example, if a current or potential medical/health trouble (e.g., high level of alcohol, acetone for diabetes, nitric oxide for asthma, etc.) is detected with the user, as determined by sampling and evaluation logic 207, messaging logic 209 may automatically generate a message (e.g., alert, note, emergency warning, routine health data, etc.) including any relevant data to be communicated as, for example, “red alert” to message/warning engine 255 of central computer over network 240 and via communication/compatibility logic 215 and communication logic 257. Similarly, a message may also be played for the user on personal device 100 via one or more output components 223.

The message received at central computer 250 from personal device 100 may be further evaluated by monitoring and evaluation engine 251 of central monitoring system 251 by, for example, comparing the information contained in the message against the user's medical history, preferences, etc., stored and maintained at database 245. Further, in one embodiment, monitoring and evaluation engine 251 may check and weigh the information contained in the message in light of any real-time changing conditions, such as detecting the possibility of a medical condition of the user which may not have been disclosed in the medical history and/or knowing the user's current location (e.g., an area with an outbreak) as facilitated by environment/location engine 255 and obtained from location and mapping logic 211, etc.

This evaluation of the message may trigger message/warning engine 255 to generate another message with more or less information and forwarded it on to one or more staff devices 270, such as to the user's primary doctor's smartphone, etc., so that proper actions may be taken in light of the findings by personal device 100 and/or central computer 250. In contrast, in some embodiments, upon further evaluation by monitoring and evaluation engine 251, the message from personal device 100 may be ignored and not forwarded on to staff devices 280, such as in case of false alarms, redundant/repeated data, etc.

At staff devices 270, in one embodiment, messages may be entered by or presented to various medical personnel (e.g., nurses, doctors, paramedics, etc.) via user interface 277 (e.g., website, software application-based user interface, etc.) as provided by software application 271. In one embodiment, any messages communicated from central computer 250 or directly from personal device 100 may be received via communication logic 275, over network 240, and viewed via user interface 277 as facilitated by message generation and presentation logic 273. Similarly, any message generated at staff devices 270 may be generated using user interface 287 as facilitated by message generation and presentation logic 273 and communicated back to central computer 250 and/or personal device 100 via communication logic 275 over network 240.

It is contemplated that messaging logic 209 may not only include a transmission module for facilitating transmission of messages, but also a reception module for reception of messages, such as warnings, alerts, notes, reminders, etc., from central monitoring system 251 at central computer 250. For example and in one embodiment, location and mapping logic 211 may work with a local GPS of capturing/sensing components 221 to continuously gather data relating to the location personal device 100 and provide this data to environment/location engine 253 of central computer 250 which, in turn, works with monitoring and evaluation engine 251 to continuously track the whereabouts of personal device 100, and thus the user, to detect any unhealthy environment, dangerous location, etc. For example, if personal device 100 is determined to be in a location where there has been an outbreak of a virus, etc., message/warning engine 255 of central computer 250 may generate a warning message which may then be communicated to the user via messaging logic 209 of personal device 100 over network 240. Further, the local GPS may continue to work with both location and mapping logic 211 to go on capturing the real-time location personal device 100 and, in turn, allowing messaging logic 209 and/or environment/location engine 255 to for any quick responses, signals, and warnings, etc., in case of a dire health condition, an unhealthy location, etc.

In one embodiment, breath testing mechanism 110 may further include energy harvesting logic 213 to work with one or more power sources and management components 225 to accept and manage any number and type of power devices and components, such as rechargeable batteries, wireless rechargeable plates, and any other energy/power sources, to ensure proper management and supply of power for personal device 100 and breath testing mechanism 110.

Capturing/sensing components 221 may include any number and type of capturing/sensing devices, such as one or more sending and/or capturing devices (e.g., cameras (e.g., three-dimension (3D) cameras, etc.), microphones, vibration components, tactile components, conductance elements, biometric sensors, chemical detectors, signal detectors, wave detectors, force sensors (e.g., accelerometers, gyroscopes), illuminators, etc.) that may be used for capturing any amount and type of visual data, such as images (e.g., photos, videos, movies, audio/video streams, etc.), and non-visual data, such as audio streams (e.g., sound, noise, vibration, ultrasound, etc.), radio waves (e.g., wireless signals, such as wireless signals having data, metadata, signs, etc.), chemical changes or properties (e.g., humidity, body temperature, etc.), biometric readings (e.g., figure prints, etc.), environmental/weather conditions, maps, etc. It is contemplated that “sensor” and “detector” may be referenced interchangeably throughout this document. It is further contemplated that one or more capturing/sensing components 221 may further include one or more supporting or supplemental devices for capturing and/or sensing of data, such as illuminators (e.g., infrared (IR) illuminator), light fixtures, generators, sound blockers, etc.

It is further contemplated that in one embodiment, capturing/sensing components 221 may further include any number and type of sensing devices or sensors (e.g., linear accelerometer) for sensing or detecting any number and type of contexts (e.g., estimating horizon, linear acceleration, etc., relating to a mobile computing device, etc.). For example, capturing/sensing components 221 may include any number and type of sensors, such as (without limitations): accelerometers (e.g., linear accelerometer to measure linear acceleration, etc.); inertial devices (e.g., inertial accelerometers, inertial gyroscopes, micro-electro-mechanical systems (MEMS) gyroscopes, inertial navigators, etc.); gravity gradiometers to study and measure variations in gravitation acceleration due to gravity, etc.

For example, capturing/sensing components 221 may further include (without limitations): audio/visual devices (e.g., cameras, microphones, speakers, etc.); context-aware sensors (e.g., temperature sensors, facial expression and feature measurement sensors working with one or more cameras of audio/visual devices, environment sensors (such as to sense background colors, lights, etc.), biometric sensors (such as to detect fingerprints, etc.), calendar maintenance and reading device), etc.; global positioning system (GPS) sensors; resource requestor; and trusted execution environment (TEE) logic. TEE logic may be employed separately or be part of resource requestor and/or an I/O subsystem, etc. Capturing/sensing components 221 may further include voice recognition devices, photo recognition devices, facial and other body recognition components, voice-to-text conversion components, etc.

Personal device 100 may further include one or more output components 223 to remain in communication with one or more capturing/sensing components 221 and one or more components of breath testing mechanism 110 to facilitate displaying of images, playing or visualization of sounds, displaying visualization of fingerprints, presenting visualization of touch, smell, and/or other sense-related experiences, etc. For example and in one embodiment, output components 223 may include (without limitation) one or more of light sources, display devices and/or screens (e.g., two-dimension (2D) displays, 3D displays, etc.), audio speakers, tactile components, conductance elements, bone conducting speakers, olfactory or smell visual and/or non/visual presentation devices, haptic or touch visual and/or non-visual presentation devices, animation display devices, biometric display devices, X-ray display devices, etc.

In the illustrated embodiment, personal device 100 is shown as hosting breath testing mechanism 110; however, it is contemplated that embodiments are not limited as such and that in another embodiment, breath testing mechanism 110 may be entirely or partially hosted by multiple or a combination of computing devices, such as computing devices 100, 250; however, throughout this document, for the sake of brevity, clarity, and ease of understanding, breath testing mechanism 100 is shown as being hosted by personal device 100.

In the illustrated embodiment, personal device 100 and staff devices 270 may include wearable devices employing one or more software applications (e.g., device applications, hardware components applications, business/social application, websites, etc.), such as software application 271, that may remain in communication with breath testing mechanism 110, where a software application may offer one or more user interfaces (e.g., web user interface (WUI), graphical user interface (GUI), touchscreen, etc.), such as user interface 277, to work with and/or facilitate one or more operations or functionalities of breath testing mechanism 110, such as displaying one or more images, videos, etc., playing one or more sounds, etc., via one or more input/output sources 108.

In one embodiment, personal and staff devices 100, 270 may include one or more of smartphones and tablet computers that their corresponding users may carry in their hands. In another embodiment, personal and staff devices 100, 270 may include toothbrushes or wearable devices, such as one or more of wearable glasses, binoculars, watches, bracelets, etc., that their corresponding users may hold in their hands or wear on their bodies, etc. In yet another embodiment, personal and staff devices 100, 270 may include other forms of wearable devices, such as one or more of clothing items, flexible wraparound wearable devices, etc., that may be of any shape or form that their corresponding users may be able to wear on their various body parts, such as knees, arms, wrists, hands, etc.

Communication/compatibility logic 215 may be used to facilitate dynamic communication and compatibility between computing device 100 and computing devices 250, 270 and any number and type of other computing devices (such as wearable computing devices, mobile computing devices, desktop computers, server computing devices, etc.), processing devices (e.g., central processing unit (CPU), graphics processing unit (GPU), etc.), capturing/sensing components 221 (e.g., non-visual data sensors/detectors, such as audio sensors, olfactory sensors, haptic sensors, signal sensors, vibration sensors, chemicals detectors, radio wave detectors, force sensors, weather/temperature sensors, body/biometric sensors, scanners, etc., and visual data sensors/detectors, such as cameras, etc.), user/context-awareness components and/or identification/verification sensors/devices (such as biometric sensors/detectors, scanners, etc.), memory or storage devices, data sources, and/or database(s) 245 (such as data storage devices, hard drives, solid-state drives, hard disks, memory cards or devices, memory circuits, etc.), network(s) 240 (e.g., Cloud network, the Internet, intranet, cellular network, proximity networks, such as Bluetooth, Bluetooth low energy (BLE), Bluetooth Smart, Wi-Fi proximity, Radio Frequency Identification (RFID), Near Field Communication (NFC), Body Area Network (BAN), etc.), wireless or wired communications and relevant protocols (e.g., Wi-Fi®, WiMAX, Ethernet, etc.), connectivity and location management techniques, software applications/websites, (e.g., social and/or business networking websites, business applications, games and other entertainment applications, etc.), programming languages, etc., while ensuring compatibility with changing technologies, parameters, protocols, standards, etc.

Throughout this document, terms like “logic”, “component”, “module”, “framework”, “engine”, “tool”, and the like, may be referenced interchangeably and include, by way of example, software, hardware, and/or any combination of software and hardware, such as firmware. Further, any use of a particular brand, word, term, phrase, name, and/or acronym, such as “personal device”, “smart device”, “staff device”, “central computer”, “toothbrush”, “mobile computer”, “wearable device”, “message”, “proximity”, “breath”, “air”, “alveolar”, “capnography”, “relative humidity”, “inhale”, “exhale”, etc., should not be read to limit embodiments to software or devices that carry that label in products or in literature external to this document.

It is contemplated that any number and type of components may be added to and/or removed from breath testing mechanism 110 to facilitate various embodiments including adding, removing, and/or enhancing certain features. For brevity, clarity, and ease of understanding of breath testing mechanism 110, many of the standard and/or known components, such as those of a computing device, are not shown or discussed here. It is contemplated that embodiments, as described herein, are not limited to any particular technology, topology, system, architecture, and/or standard and are dynamic enough to adopt and adapt to any future changes.

FIG. 2B illustrates an architectural placement 280 of a selective set of components of dynamic breath testing mechanism 110 of FIGS. 1-2A according to one embodiment. For brevity, many of the details discussed with reference to FIGS. 1 and 2A may not be discussed or repeated hereafter. It is contemplated and to be noted that embodiments are not limited to the illustrated architectural placement, whether it be in terms of the illustrated components or their placement, and that this placement is merely provided as an example for brevity, clarity, and ease understanding.

As illustrated and in one embodiment, architectural placement 280 includes personal device 100 having a set of components including one or more of (without limitation): capturing/sensing components 221 (e.g., GPS and inertial sensors (e.g., accelerometer, gyroscope, etc.)); power sources and management components 225 (e.g., battery/energy/power harvesting components); microcontroller for processing fusing data 281, low power radio module (e.g., WiFi, Long-Term Evolution (LTE), etc.), and breath testing mechanism 110.

FIG. 2C illustrates a personal device 100 according to one embodiment. As previously discussed with reference to FIG. 2B, personal device 100 may include any number of smart devices, such as a smart toothbrush, as illustrated, having architectural placement 280 of FIG. 2B.

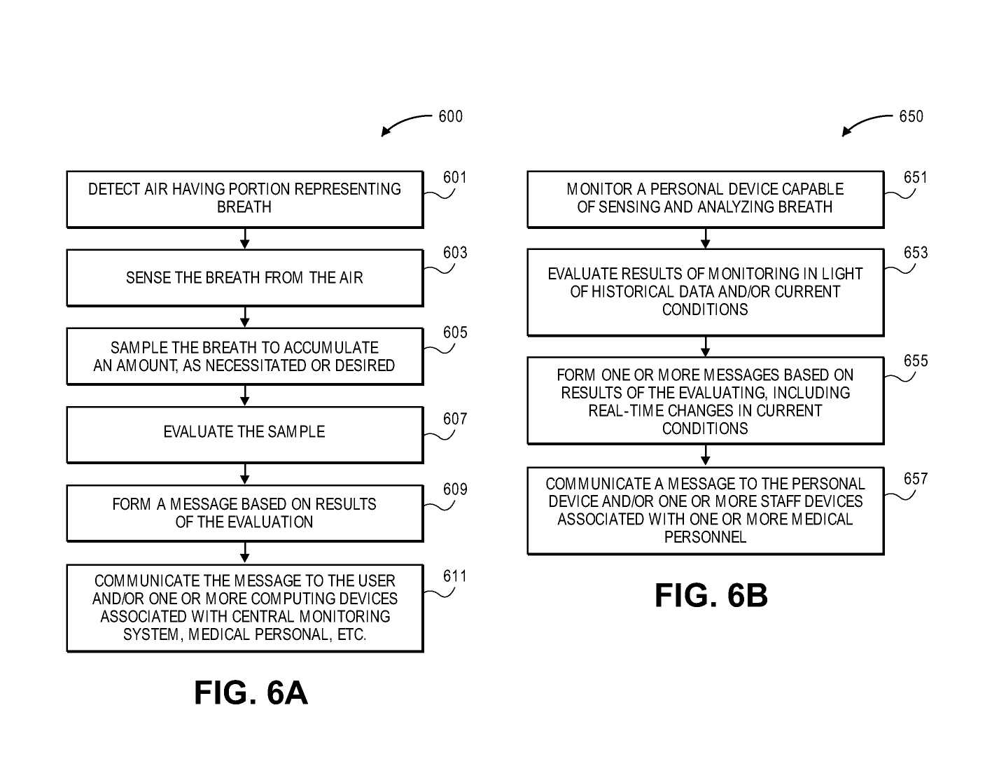

Referring now to FIG. 6A, it illustrates a method 600 for performing breath testing tasks according to one embodiment. Method 600 may be performed by processing logic that may comprise hardware (e.g., circuitry, dedicated logic, programmable logic, etc.), software (such as instructions run on a processing device), or a combination thereof. In one embodiment, method 600 may be performed by breath testing mechanism 110 of FIGS. 1-2A. The processes of method 600 are illustrated in linear sequences for brevity and clarity in presentation; however, it is contemplated that any number of them can be performed in parallel, asynchronously, or in different orders. For brevity, many of the details discussed with reference to the previous figures may not be discussed or repeated hereafter.

Method 600 begins at block 601 with detection of air having portions containing breath, such as alveolar breath, at a personal device (e.g., smart toothbrush). At block 603, this breath or these portions of the air having the breath are sensed. At block 605, the breath is sampled to accumulate an amount of breath, as necessitated or desired depending on the type of test that is being performed, such as detecting alcohol level, asthma, diabetes, etc., or simply performing a routine test, etc. At block 607, the sample is analyzed and evaluated based on the type of test being performed. At block 609, depending on the results of the evaluation, one or more messages (e.g., note, warning, signal, alert, etc.) may be generated. At block 611, in one embodiment, a message may be provided to the user of the personal device so that the user may view any relevant information about the breath test and similarly, in one embodiment, the same or a variation of the message may be communicated over to a central monitoring system at a central computer so that it may be further evaluated and forwarded on to one or more computing devices (e.g., smartphone, tablet computer, etc.) associated with one or more medical personnel (e.g., doctor, first response, etc.) associated with the user or as necessitated by the evaluation results.

FIG. 6B illustrates a method 650 for monitoring and evaluation of personal devices and users in relation to breath testing according to one embodiment. Method 650 may be performed by processing logic that may comprise hardware (e.g., circuitry, dedicated logic, programmable logic, etc.), software (such as instructions run on a processing device), or a combination thereof. In one embodiment, method 650 may be performed by central monitoring system 251 of FIG. 2A. The processes of method 650 are illustrated in linear sequences for brevity and clarity in presentation; however, it is contemplated that any number of them can be performed in parallel, asynchronously, or in different orders. For brevity, many of the details discussed with reference to the previous figures may not be discussed or repeated hereafter.

Method 650 begins at block 651 with monitoring a personal device (e.g., smart toothbrush) associated with a user, where the personal device is capable of performing breath testing on the user as facilitated by breath testing mechanism 110 of FIGS. 1-2A. At block 653, any results of the monitoring are evaluated in light of current conditions and/or any historical data relating to the user's health, the personal device, etc. At block 655, in one embodiment, based on results of the evaluation, one or more messages may be formed. At block 657, a message, such as a warning, may be communicated back to the user via the personal device and/or another message, the same or somewhat varying message, may be provided to one or more computing devices (e.g., smartphone, tablet computer, etc.) associated with one or more medical personnel (e.g., doctor, first response, etc.) associated with the user so that any necessary actions may be taken by the medical personnel.

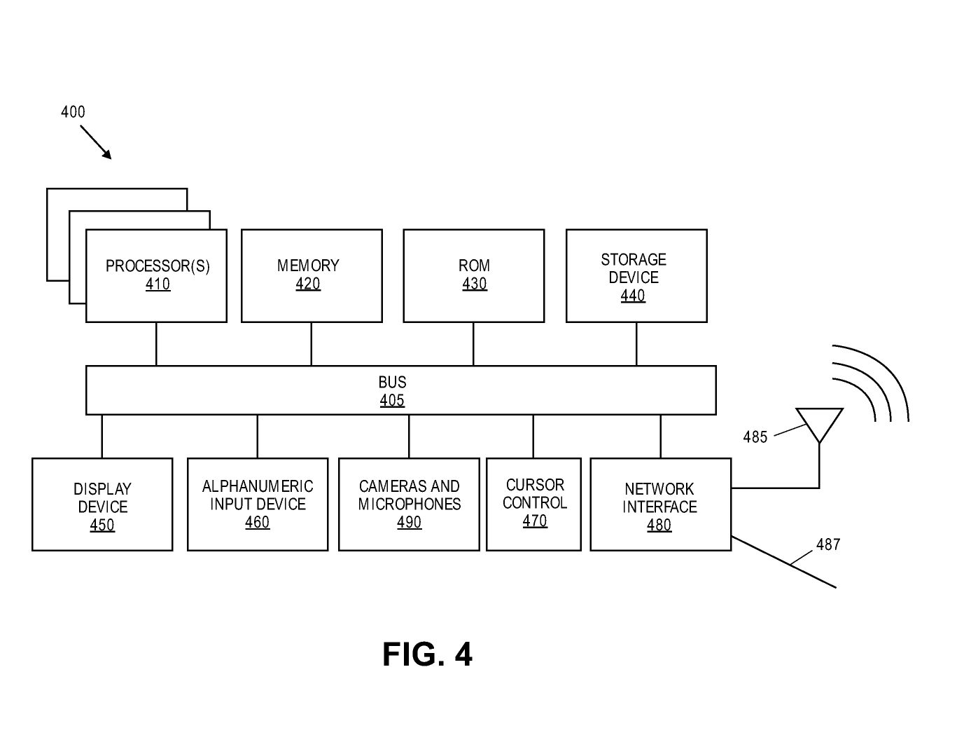

Now referring to FIG. 4, it illustrates an embodiment of a computing system 400 capable of supporting the operations discussed above. Computing system 400 represents a range of computing and electronic devices (wired or wireless) including, for example, desktop computing systems, laptop computing systems, cellular telephones, personal digital assistants (PDAs) including cellular-enabled PDAs, set top boxes, smartphones, tablets, wearable devices, etc. Alternate computing systems may include more, fewer and/or different components. Computing device 400 may be the same as or similar to or include computing devices 100 described in reference to FIG. 1.

Computing system 400 includes bus 405 (or, for example, a link, an interconnect, or another type of communication device or interface to communicate information) and processor 410 coupled to bus 405 that may process information. While computing system 400 is illustrated with a single processor, it may include multiple processors and/or co-processors, such as one or more of central processors, image signal processors, graphics processors, and vision processors, etc. Computing system 400 may further include random access memory (RAM) or other dynamic storage device 420 (referred to as main memory), coupled to bus 405 and may store information and instructions that may be executed by processor 410. Main memory 420 may also be used to store temporary variables or other intermediate information during execution of instructions by processor 410.

Computing system 400 may also include read only memory (ROM) and/or other storage device 430 coupled to bus 405 that may store static information and instructions for processor 410. Date storage device 440 may be coupled to bus 405 to store information and instructions. Date storage device 440, such as magnetic disk or optical disc and corresponding drive may be coupled to computing system 400.

Computing system 400 may also be coupled via bus 405 to display device 450, such as a cathode ray tube (CRT), liquid crystal display (LCD) or Organic Light Emitting Diode (OLED) array, to display information to a user. User input device 460, including alphanumeric and other keys, may be coupled to bus 405 to communicate information and command selections to processor 410. Another type of user input device 460 is cursor control 470, such as a mouse, a trackball, a touchscreen, a touchpad, or cursor direction keys to communicate direction information and command selections to processor 410 and to control cursor movement on display 450. Camera and microphone arrays 490 of computer system 400 may be coupled to bus 405 to observe gestures, record audio and video and to receive and transmit visual and audio commands.

Computing system 400 may further include network interface(s) 480 to provide access to a network, such as a local area network (LAN), a wide area network (WAN), a metropolitan area network (MAN), a personal area network (PAN), Bluetooth, a cloud network, a mobile network (e.g., 3^{rd} Generation (3G), etc.), an intranet, the Internet, etc. Network interface(s) 480 may include, for example, a wireless network interface having antenna 485, which may represent one or more antenna(e). Network interface(s) 480 may also include, for example, a wired network interface to communicate with remote devices via network cable 487, which may be, for example, an Ethernet cable, a coaxial cable, a fiber optic cable, a serial cable, or a parallel cable.

Network interface(s) 480 may provide access to a LAN, for example, by conforming to IEEE 802.11b and/or IEEE 802.11g standards, and/or the wireless network interface may provide access to a personal area network, for example, by conforming to Bluetooth standards. Other wireless network interfaces and/or protocols, including previous and subsequent versions of the standards, may also be supported.

In addition to, or instead of, communication via the wireless LAN standards, network interface(s) 480 may provide wireless communication using, for example, Time Division, Multiple Access (TDMA) protocols, Global Systems for Mobile Communications (GSM) protocols, Code Division, Multiple Access (CDMA) protocols, and/or any other type of wireless communications protocols.

Network interface(s) 480 may include one or more communication interfaces, such as a modem, a network interface card, or other well-known interface devices, such as those used for coupling to the Ethernet, token ring, or other types of physical wired or wireless attachments for purposes of providing a communication link to support a LAN or a WAN, for example. In this manner, the computer system may also be coupled to a number of peripheral devices, clients, control surfaces, consoles, or servers via a conventional network infrastructure, including an Intranet or the Internet, for example.

It is to be appreciated that a lesser or more equipped system than the example described above may be preferred for certain implementations. Therefore, the configuration of computing system 400 may vary from implementation to implementation depending upon numerous factors, such as price constraints, performance requirements, technological improvements, or other circumstances. Examples of the electronic device or computer system 400 may include without limitation a mobile device, a personal digital assistant, a mobile computing device, a smartphone, a cellular telephone, a handset, a one-way pager, a two-way pager, a messaging device, a computer, a personal computer (PC), a desktop computer, a laptop computer, a notebook computer, a handheld computer, a tablet computer, a server, a server array or server farm, a web server, a network server, an Internet server, a work station, a mini-computer, a main frame computer, a supercomputer, a network appliance, a web appliance, a distributed computing system, multiprocessor systems, processor-based systems, consumer electronics, programmable consumer electronics, television, digital television, set top box, wireless access point, base station, subscriber station, mobile subscriber center, radio network controller, router, hub, gateway, bridge, switch, machine, or combinations thereof.

Embodiments may be implemented as any or a combination of: one or more microchips or integrated circuits interconnected using a parentboard, hardwired logic, software stored by a memory device and executed by a microprocessor, firmware, an application specific integrated circuit (ASIC), and/or a field programmable gate array (FPGA). The term “logic” may include, by way of example, software or hardware and/or combinations of software and hardware.

Embodiments may be provided, for example, as a computer program product which may include one or more machine-readable media having stored thereon machine-executable instructions that, when executed by one or more machines such as a computer, network of computers, or other electronic devices, may result in the one or more machines carrying out operations in accordance with embodiments described herein. A machine-readable medium may include, but is not limited to, floppy diskettes, optical disks, CD-ROMs (Compact Disc-Read Only Memories), and magneto-optical disks, ROMs, RAMs, EPROMs (Erasable Programmable Read Only Memories), EEPROMs (Electrically Erasable Programmable Read Only Memories), magnetic or optical cards, flash memory, or other type of media/machine-readable medium suitable for storing machine-executable instructions.

Moreover, embodiments may be downloaded as a computer program product, wherein the program may be transferred from a remote computer (e.g., a server) to a requesting computer (e.g., a client) by way of one or more data signals embodied in and/or modulated by a carrier wave or other propagation medium via a communication link (e.g., a modem and/or network connection).

References to “one embodiment”, “an embodiment”, “example embodiment”, “various embodiments”, etc., indicate that the embodiment(s) so described may include particular features, structures, or characteristics, but not every embodiment necessarily includes the particular features, structures, or characteristics. Further, some embodiments may have some, all, or none of the features described for other embodiments.

In the following description and claims, the term “coupled” along with its derivatives, may be used. “Coupled” is used to indicate that two or more elements co-operate or interact with each other, but they may or may not have intervening physical or electrical components between them.

As used in the claims, unless otherwise specified the use of the ordinal adjectives “first”, “second”, “third”, etc., to describe a common element, merely indicate that different instances of like elements are being referred to, and are not intended to imply that the elements so described must be in a given sequence, either temporally, spatially, in ranking, or in any other manner.

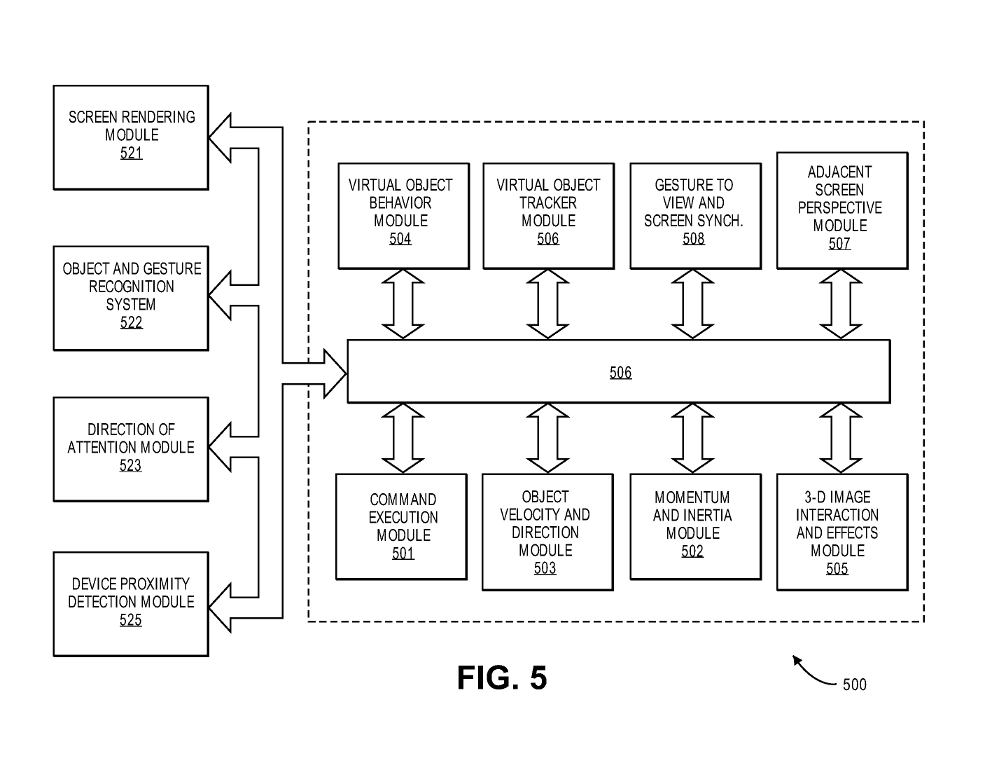

FIG. 5 illustrates an embodiment of a computing environment 500 capable of supporting the operations discussed above. The modules and systems can be implemented in a variety of different hardware architectures and form factors including that shown in FIG. 4.

The Command Execution Module 501 includes a central processing unit to cache and execute commands and to distribute tasks among the other modules and systems shown. It may include an instruction stack, a cache memory to store intermediate and final results, and mass memory to store applications and operating systems. The Command Execution Module may also serve as a central coordination and task allocation unit for the system.

The Screen Rendering Module 521 draws objects on the one or more multiple screens for the user to see. It can be adapted to receive the data from the Virtual Object Behavior Module 504, described below, and to render the virtual object and any other objects and forces on the appropriate screen or screens. Thus, the data from the Virtual Object Behavior Module would determine the position and dynamics of the virtual object and associated gestures, forces and objects, for example, and the Screen Rendering Module would depict the virtual object and associated objects and environment on a screen, accordingly. The Screen Rendering Module could further be adapted to receive data from the Adjacent Screen Perspective Module 507, described below, to either depict a target landing area for the virtual object if the virtual object could be moved to the display of the device with which the Adjacent Screen Perspective Module is associated. Thus, for example, if the virtual object is being moved from a main screen to an auxiliary screen, the Adjacent Screen Perspective Module 2 could send data to the Screen Rendering Module to suggest, for example in shadow form, one or more target landing areas for the virtual object on that track to a user's hand movements or eye movements.

The Object and Gesture Recognition System 522 may be adapted to recognize and track hand and harm gestures of a user. Such a module may be used to recognize hands, fingers, finger gestures, hand movements and a location of hands relative to displays. For example, the Object and Gesture Recognition Module could for example determine that a user made a body part gesture to drop or throw a virtual object onto one or the other of the multiple screens, or that the user made a body part gesture to move the virtual object to a bezel of one or the other of the multiple screens. The Object and Gesture Recognition System may be coupled to a camera or camera array, a microphone or microphone array, a touch screen or touch surface, or a pointing device, or some combination of these items, to detect gestures and commands from the user.

The touch screen or touch surface of the Object and Gesture Recognition System may include a touch screen sensor. Data from the sensor may be fed to hardware, software, firmware or a combination of the same to map the touch gesture of a user's hand on the screen or surface to a corresponding dynamic behavior of a virtual object. The sensor date may be used to momentum and inertia factors to allow a variety of momentum behavior for a virtual object based on input from the user's hand, such as a swipe rate of a user's finger relative to the screen. Pinching gestures may be interpreted as a command to lift a virtual object from the display screen, or to begin generating a virtual binding associated with the virtual object or to zoom in or out on a display. Similar commands may be generated by the Object and Gesture Recognition System using one or more cameras without benefit of a touch surface.

The Direction of Attention Module 523 may be equipped with cameras or other sensors to track the position or orientation of a user's face or hands. When a gesture or voice command is issued, the system can determine the appropriate screen for the gesture. In one example, a camera is mounted near each display to detect whether the user is facing that display. If so, then the direction of attention module information is provided to the Object and Gesture Recognition Module 522 to ensure that the gestures or commands are associated with the appropriate library for the active display. Similarly, if the user is looking away from all of the screens, then commands can be ignored.

The Device Proximity Detection Module 525 can use proximity sensors, compasses, GPS (global positioning system) receivers, personal area network radios, and other types of sensors, together with triangulation and other techniques to determine the proximity of other devices. Once a nearby device is detected, it can be registered to the system and its type can be determined as an input device or a display device or both. For an input device, received data may then be applied to the Object Gesture and Recognition System 522. For a display device, it may be considered by the Adjacent Screen Perspective Module 507.

The Virtual Object Behavior Module 504 is adapted to receive input from the Object Velocity and Direction Module, and to apply such input to a virtual object being shown in the display. Thus, for example, the Object and Gesture Recognition System would interpret a user gesture and by mapping the captured movements of a user's hand to recognized movements, the Virtual Object Tracker Module would associate the virtual object's position and movements to the movements as recognized by Object and Gesture Recognition System, the Object and Velocity and Direction Module would capture the dynamics of the virtual object's movements, and the Virtual Object Behavior Module would receive the input from the Object and Velocity and Direction Module to generate data that would direct the movements of the virtual object to correspond to the input from the Object and Velocity and Direction Module.

The Virtual Object Tracker Module 506 on the other hand may be adapted to track where a virtual object should be located in three dimensional space in a vicinity of an display, and which body part of the user is holding the virtual object, based on input from the Object and Gesture Recognition Module. The Virtual Object Tracker Module 506 may for example track a virtual object as it moves across and between screens and track which body part of the user is holding that virtual object. Tracking the body part that is holding the virtual object allows a continuous awareness of the body part's air movements, and thus an eventual awareness as to whether the virtual object has been released onto one or more screens.

The Gesture to View and Screen Synchronization Module 508, receives the selection of the view and screen or both from the Direction of Attention Module 523 and, in some cases, voice commands to determine which view is the active view and which screen is the active screen. It then causes the relevant gesture library to be loaded for the Object and Gesture Recognition System 522. Various views of an application on one or more screens can be associated with alternative gesture libraries or a set of gesture templates for a given view. As an example in FIG. 1A a pinch-release gesture launches a torpedo, but in FIG. 1B, the same gesture launches a depth charge.

The Adjacent Screen Perspective Module 507, which may include or be coupled to the Device Proximity Detection Module 525, may be adapted to determine an angle and position of one display relative to another display. A projected display includes, for example, an image projected onto a wall or screen. The ability to detect a proximity of a nearby screen and a corresponding angle or orientation of a display projected therefrom may for example be accomplished with either an infrared emitter and receiver, or electromagnetic or photo-detection sensing capability. For technologies that allow projected displays with touch input, the incoming video can be analyzed to determine the position of a projected display and to correct for the distortion caused by displaying at an angle. An accelerometer, magnetometer, compass, or camera can be used to determine the angle at which a device is being held while infrared emitters and cameras could allow the orientation of the screen device to be determined in relation to the sensors on an adjacent device. The Adjacent Screen Perspective Module 507 may, in this way, determine coordinates of an adjacent screen relative to its own screen coordinates. Thus, the Adjacent Screen Perspective Module may determine which devices are in proximity to each other, and further potential targets for moving one or more virtual object's across screens. The Adjacent Screen Perspective Module may further allow the position of the screens to be correlated to a model of three-dimensional space representing all of the existing objects and virtual objects.

The Object and Velocity and Direction Module 503 may be adapted to estimate the dynamics of a virtual object being moved, such as its trajectory, velocity (whether linear or angular), momentum (whether linear or angular), etc. by receiving input from the Virtual Object Tracker Module. The Object and Velocity and Direction Module may further be adapted to estimate dynamics of any physics forces, by for example estimating the acceleration, deflection, degree of stretching of a virtual binding, etc. and the dynamic behavior of a virtual object once released by a user's body part. The Object and Velocity and Direction Module may also use image motion, size and angle changes to estimate the velocity of objects, such as the velocity of hands and fingers

The Momentum and Inertia Module 502 can use image motion, image size, and angle changes of objects in the image plane or in a three-dimensional space to estimate the velocity and direction of objects in the space or on a display. The Momentum and Inertia Module is coupled to the Object and Gesture Recognition System 522 to estimate the velocity of gestures performed by hands, fingers, and other body parts and then to apply those estimates to determine momentum and velocities to virtual objects that are to be affected by the gesture.

The 3D Image Interaction and Effects Module 505 tracks user interaction with 3D images that appear to extend out of one or more screens. The influence of objects in the z-axis (towards and away from the plane of the screen) can be calculated together with the relative influence of these objects upon each other. For example, an object thrown by a user gesture can be influenced by 3D objects in the foreground before the virtual object arrives at the plane of the screen. These objects may change the direction or velocity of the projectile or destroy it entirely. The object can be rendered by the 3D Image Interaction and Effects Module in the foreground on one or more of the displays.

The following clauses and/or examples pertain to further embodiments or examples. Specifics in the examples may be used anywhere in one or more embodiments. The various features of the different embodiments or examples may be variously combined with some features included and others excluded to suit a variety of different applications. Examples may include subject matter such as a method, means for performing acts of the method, at least one machine-readable medium including instructions that, when performed by a machine cause the machine to performs acts of the method, or of an apparatus or system for facilitating hybrid communication according to embodiments and examples described herein.

Some embodiments pertain to Example 1 that includes an apparatus to facilitate dynamic and seamless breath testing at computing devices, comprising: detection logic to detect air exhaled by a user into a first computing device, wherein the air includes breath associated with the user; sensing logic to sense the breath in the air; sampling and evaluation logic to obtain a sample of the breath, and evaluate the sample; messaging logic to generate a message based on the evaluation of the sample; and communication/compatibility logic to present, via one or more output components, the message to the user via a user interface, wherein the message includes results of the evaluation of the breath sample.

Example 2 includes the subject matter of Example 1, wherein the message comprises one or more of a brief overview of the user's health, a detailed analysis of the breath, a warning, an alert, a note, a reminder, and a conflict, wherein the message is presented in one or more forms including one or more of an audio message, a video message, an image message, a olfactory message, and a haptic message.

Example 3 includes the subject matter of Example 1, wherein one or more portions of the air represent the breath including alveolar breath, wherein the sensing logic is further to sense the breath based on determination of one or more of concentration of carbon dioxide in the alveolar breath and relative humidity in the breath.

Example 4 includes the subject matter of Example 1, further comprising identification/authentication logic to identify and authenticate at least one of the first computing device and the user.

Example 5 includes the subject matter of Example 1 or 4, wherein the first computing device comprises a smart toothbrush.

Example 6 includes the subject matter of Example 1 or 4, wherein the first computing device further comprises smart mobile computers including one or more of smartphones, tablet computers, head-mounted displays, head-mounted gaming displays, wearable glasses, wearable binoculars, smart jewelry, smartwatches, smartcards, and smart clothing items.

Example 7 includes the subject matter of Example 1, further comprising energy harvesting logic to manage one or more power sources associated with the first computing device, wherein managing includes ensuring sufficient power supply to the first computing device from the one or more power sources, the one or more power sources having at least one of a rechargeable battery and a wireless charging plate.

Example 8 includes the subject matter of Example 1, further comprising location and mapping logic to determine, in real-time, one or more locations associated with the first computing device, wherein the location and mapping logic is further to communicate, in real-time, the one or more locations to a second computing device, wherein the second computing device includes a server computer.

Example 9 includes the subject matter of Example 8, wherein the location and mapping logic is further to continuously receive, via communication/compatibility logic, one or more notices relating to changing conditions associated with the one or more locations, wherein the one or more notices include a warning indicating an occurrence a dire condition associated with a location of the one or more locations.

Example 10 includes the subject matter of Example 9, wherein the warning is further communicated to one or more computing devices associated with one or more medical personnel, wherein the one or more computing devices include one or more of desktop computers and mobile computers including one or more of smartphones, tablet computers, head-mounted displays, head-mounted gaming displays, wearable glasses, wearable binoculars, smart jewelry, smartwatches, smartcards, and smart clothing items.

Some embodiments pertain to Example 11 that includes a method for dynamically facilitating dynamic and seamless breath testing at computing devices, comprising: detecting air exhaled by a user into a first computing device, wherein the air includes breath associated with the user; sensing the breath in the air; obtaining a sample of the breath, and evaluating the sample; generating a message based on the evaluation of the sample; and presenting, via one or more output components, the message to the user via a user interface, wherein the message includes results of the evaluation of the breath sample.

Example 12 includes the subject matter of Example 11, wherein the message comprises one or more of a brief overview of the user's health, a detailed analysis of the breath, a warning, an alert, a note, a reminder, and a conflict, wherein the message is presented in one or more forms including one or more of an audio message, a video message, an image message, a olfactory message, and a haptic message.

Example 13 includes the subject matter of Example 11, wherein one or more portions of the air represent the breath including alveolar breath, wherein the sensing logic is further to sense the breath based on determination of one or more of concentration of carbon dioxide in the alveolar breath and relative humidity in the breath.

Example 14 includes the subject matter of Example 11, further comprising identifying and authenticating at least one of the first computing device and the user.

Example 15 includes the subject matter of Example 11 or 14, wherein the first computing device comprises a smart toothbrush.

Example 16 includes the subject matter of Example 11 or 14, wherein the first computing device further comprises smart mobile computers including one or more of smartphones, tablet computers, head-mounted displays, head-mounted gaming displays, wearable glasses, wearable binoculars, smart jewelry, smartwatches, smartcards, and smart clothing items.

Example 17 includes the subject matter of Example 11, further comprising managing one or more power sources associated with the first computing device, wherein managing includes ensuring sufficient power supply to the first computing device from the one or more power sources, the one or more power sources having at least one of a rechargeable battery and a wireless charging plate.

Example 18 includes the subject matter of Example 11, further comprising: determining, in real-time, one or more locations associated with the first computing device; and communicating, in real-time, the one or more locations to a second computing device, wherein the second computing device includes a server computer.

Example 19 includes the subject matter of Example 18, further comprising continuously receiving one or more notices relating to changing conditions associated with the one or more locations, wherein the one or more notices include a warning indicating an occurrence a dire condition associated with a location of the one or more locations.

Example 20 includes the subject matter of Example 19, wherein the warning is further communicated to one or more computing devices associated with one or more medical personnel, wherein the one or more computing devices include one or more of desktop computers and mobile computers including one or more of smartphones, tablet computers, head-mounted displays, head-mounted gaming displays, wearable glasses, wearable binoculars, smart jewelry, smartwatches, smartcards, and smart clothing items.

Example 21 includes at least one machine-readable medium comprising a plurality of instructions, when executed on a computing device, to implement or perform a method or realize an apparatus as claimed in any preceding claims or embodiments or examples.

Example 22 includes at least one non-transitory or tangible machine-readable medium comprising a plurality of instructions, when executed on a computing device, to implement or perform a method or realize an apparatus as claimed in any preceding claims or examples.

Example 23 includes a system comprising a mechanism to implement or perform a method or realize an apparatus as claimed in any preceding claims or embodiments or examples.

Example 24 includes an apparatus comprising means to perform a method as claimed in any preceding claims or embodiments or examples.

Example 25 includes a computing device arranged to implement or perform a method or realize an apparatus as claimed in any preceding claims or embodiments or examples.

Example 26 includes a communications device arranged to implement or perform a method or realize an apparatus as claimed in any preceding claims or embodiments or examples.

Some embodiments pertain to Example 27 includes a system comprising a storage device having instructions, and a processor to execute the instructions to facilitate a mechanism to perform one or more operations comprising: detecting air exhaled by a user into a first computing device, wherein the air includes breath associated with the user; sensing the breath in the air; obtaining a sample of the breath, and evaluating the sample; generating a message based on the evaluation of the sample; and presenting, via one or more output components, the message to the user via a user interface, wherein the message includes results of the evaluation of the breath sample.

Example 28 includes the subject matter of Example 27, wherein the message comprises one or more of a brief overview of the user's health, a detailed analysis of the breath, a warning, an alert, a note, a reminder, and a conflict, wherein the message is presented in one or more forms including one or more of an audio message, a video message, an image message, a olfactory message, and a haptic message.

Example 29 includes the subject matter of Example 27, wherein one or more portions of the air represent the breath including alveolar breath, wherein the sensing logic is further to sense the breath based on determination of one or more of concentration of carbon dioxide in the alveolar breath and relative humidity in the breath.

Example 30 includes the subject matter of Example 27, wherein the one or more operations further comprise identifying and authenticating at least one of the first computing device and the user.

Example 31 includes the subject matter of Example 27 or 30, wherein the first computing device comprises a smart toothbrush.

Example 32 includes the subject matter of Example 27 or 30, wherein the first computing device further comprises smart mobile computers including one or more of smartphones, tablet computers, head-mounted displays, head-mounted gaming displays, wearable glasses, wearable binoculars, smart jewelry, smartwatches, smartcards, and smart clothing items.

Example 33 includes the subject matter of Example 27, wherein the one or more operations further comprise managing one or more power sources associated with the first computing device, wherein managing includes ensuring sufficient power supply to the first computing device from the one or more power sources, the one or more power sources having at least one of a rechargeable battery and a wireless charging plate.

Example 34 includes the subject matter of Example 27, wherein the one or more operations further comprise: determining, in real-time, one or more locations associated with the first computing device; and communicating, in real-time, the one or more locations to a second computing device, wherein the second computing device includes a server computer.

Example 35 includes the subject matter of Example 34, wherein the one or more operations further comprise continuously receiving one or more notices relating to changing conditions associated with the one or more locations, wherein the one or more notices include a warning indicating an occurrence a dire condition associated with a location of the one or more locations.

Example 36 includes the subject matter of Example 35, wherein the warning is further communicated to one or more computing devices associated with one or more medical personnel, wherein the one or more computing devices include one or more of desktop computers and mobile computers including one or more of smartphones, tablet computers, head-mounted displays, head-mounted gaming displays, wearable glasses, wearable binoculars, smart jewelry, smartwatches, smartcards, and smart clothing items.

Some embodiments pertain to Example 37 includes an apparatus comprising: means for detecting air exhaled by a user into a first computing device, wherein the air includes breath associated with the user; means for sensing the breath in the air; means for obtaining a sample of the breath, and evaluating the sample; means for generating a message based on the evaluation of the sample; and means for presenting, via one or more output components, the message to the user via a user interface, wherein the message includes results of the evaluation of the breath sample.

Example 38 includes the subject matter of Example 37, wherein the message comprises one or more of a brief overview of the user's health, a detailed analysis of the breath, a warning, an alert, a note, a reminder, and a conflict, wherein the message is presented in one or more forms including one or more of an audio message, a video message, an image message, a olfactory message, and a haptic message.

Example 39 includes the subject matter of Example 37, wherein one or more portions of the air represent the breath including alveolar breath, wherein the sensing logic is further to sense the breath based on determination of one or more of concentration of carbon dioxide in the alveolar breath and relative humidity in the breath.

Example 40 includes the subject matter of Example 37, further comprising means for identifying and authenticating at least one of the first computing device and the user.

Example 41 includes the subject matter of Example 37 or 40, wherein the first computing device comprises a smart toothbrush.

Example 42 includes the subject matter of Example 37 or 40, wherein the first computing device further comprises smart mobile computers including one or more of smartphones, tablet computers, head-mounted displays, head-mounted gaming displays, wearable glasses, wearable binoculars, smart jewelry, smartwatches, smartcards, and smart clothing items.

Example 43 includes the subject matter of Example 37, further comprising means for managing one or more power sources associated with the first computing device, wherein managing includes ensuring sufficient power supply to the first computing device from the one or more power sources, the one or more power sources having at least one of a rechargeable battery and a wireless charging plate.

Example 44 includes the subject matter of Example 37, further comprising: means for determining, in real-time, one or more locations associated with the first computing device; and means for communicating, in real-time, the one or more locations to a second computing device, wherein the second computing device includes a server computer.

Example 45 includes the subject matter of Example 44, further comprising means for continuously receiving one or more notices relating to changing conditions associated with the one or more locations, wherein the one or more notices include a warning indicating an occurrence a dire condition associated with a location of the one or more locations.

Example 46 includes the subject matter of Example 45, wherein the warning is further communicated to one or more computing devices associated with one or more medical personnel, wherein the one or more computing devices include one or more of desktop computers and mobile computers including one or more of smartphones, tablet computers, head-mounted displays, head-mounted gaming displays, wearable glasses, wearable binoculars, smart jewelry, smartwatches, smartcards, and smart clothing items.

Some embodiments pertain to Example 47 includes an apparatus comprising: detection logic to detect saliva in a mouth of a user accessing a first computing device; sensing logic to sense ingredients or components in the saliva; sampling and evaluation logic to obtain a sample of the saliva, and evaluate the saliva; messaging logic to generate a message based on the evaluation of the sample; and communication/compatibility logic to present, via one or more output components, the message to the user via a user interface, wherein the message includes results of the evaluation of the saliva sample.

Example 48 includes the subject matter of Example 47, wherein the message comprises one or more of a brief overview of the user's health, a detailed analysis of the saliva, a warning, an alert, a note, a reminder, and a conflict, wherein the message is presented in one or more forms including one or more of an audio message, a video message, an image message, a olfactory message, and a haptic message.

Example 49 includes the subject matter of Example 47, wherein the sensing logic is further to sense the saliva based on determination of one or more of concentration of carbon dioxide, relative humidity, and other harmful ingredients or components in the saliva.

Example 50 includes the subject matter of Example 47, further comprising identification/authentication logic to identify and authenticate at least one of the first computing device and the user.

Example 51 includes the subject matter of Example 50, wherein the first computing device comprises a smart toothbrush.

Example 52 includes the subject matter of Example 50, wherein the first computing device further comprises smart mobile computers including one or more of smartphones, tablet computers, head-mounted displays, head-mounted gaming displays, wearable glasses, wearable binoculars, smart jewelry, smartwatches, smartcards, and smart clothing items.

Example 53 includes the subject matter of Example 47, further comprising energy harvesting logic to manage one or more power sources associated with the first computing device, wherein managing includes ensuring sufficient power supply to the first computing device from the one or more power sources, the one or more power sources having at least one of a rechargeable battery and a wireless charging plate.

Example 54 includes the subject matter of Example 47, further comprising location and mapping logic to determine, in real-time, one or more locations associated with the first computing device, wherein the location and mapping logic is further to communicate, in real-time, the one or more locations to a second computing device, wherein the second computing device includes a server computer.

Example 55 includes the subject matter of Example 54, wherein the location and mapping logic is further to continuously receive, via communication/compatibility logic, one or more notices relating to changing conditions associated with the one or more locations, wherein the one or more notices include a warning indicating an occurrence a dire condition associated with a location of the one or more locations.

Example 56 includes the subject matter of Example 55, wherein the warning is further communicated to one or more computing devices associated with one or more medical personnel, wherein the one or more computing devices include one or more of desktop computers and mobile computers including one or more of smartphones, tablet computers, head-mounted displays, head-mounted gaming displays, wearable glasses, wearable binoculars, smart jewelry, smartwatches, smartcards, and smart clothing items.

Some embodiments pertain to Example 57 includes a method comprising: detecting saliva in a mouth of a user accessing a first computing device; sensing ingredients or components in the saliva; obtaining a sample of the saliva, and evaluating the sample; generating a message based on the evaluation of the sample; and presenting, via one or more output components, the message to the user via a user interface, wherein the message includes results of the evaluation of the saliva sample.

Example 58 includes the subject matter of Example 57, wherein the message comprises one or more of a brief overview of the user's health, a detailed analysis of the saliva, a warning, an alert, a note, a reminder, and a conflict, wherein the message is presented in one or more forms including one or more of an audio message, a video message, an image message, a olfactory message, and a haptic message.

Example 59 includes the subject matter of Example 57, further comprising sensing the saliva based on determination of one or more of concentration of carbon dioxide, relative humidity, and other harmful ingredients or components in the saliva.

Example 60 includes the subject matter of Example 57, further comprising identifying and authenticating at least one of the first computing device and the user.

Example 61 includes the subject matter of Example 60, wherein the first computing device comprises a smart toothbrush.

Example 62 includes the subject matter of Example 60, wherein the first computing device further comprises smart mobile computers including one or more of smartphones, tablet computers, head-mounted displays, head-mounted gaming displays, wearable glasses, wearable binoculars, smart jewelry, smartwatches, smartcards, and smart clothing items.

Example 63 includes the subject matter of Example 57, further comprising managing one or more power sources associated with the first computing device, wherein managing includes ensuring sufficient power supply to the first computing device from the one or more power sources, the one or more power sources having at least one of a rechargeable battery and a wireless charging plate.

Example 64 includes the subject matter of Example 57, further comprising: determining, in real-time, one or more locations associated with the first computing device; and communicating, in real-time, the one or more locations to a second computing device, wherein the second computing device includes a server computer.

Example 65 includes the subject matter of Example 64, further comprising continuously receiving one or more notices relating to changing conditions associated with the one or more locations, wherein the one or more notices include a warning indicating an occurrence a dire condition associated with a location of the one or more locations.

Example 66 includes the subject matter of Example 65, wherein the warning is further communicated to one or more computing devices associated with one or more medical personnel, wherein the one or more computing devices include one or more of desktop computers and mobile computers including one or more of smartphones, tablet computers, head-mounted displays, head-mounted gaming displays, wearable glasses, wearable binoculars, smart jewelry, smartwatches, smartcards, and smart clothing items.

Example 67 includes at least one non-transitory or tangible machine-readable medium comprising a plurality of instructions, when executed on a computing device, to implement or perform a method as claimed in any of claims or examples 11-20 or 57-66.

Example 68 includes at least one machine-readable medium comprising a plurality of instructions, when executed on a computing device, to implement or perform a method as claimed in any of claims or examples 11-20 or 57-66.

Example 69 includes a system comprising a mechanism to implement or perform a method as claimed in any of claims or examples 11-20 or 57-66.

Example 70 includes an apparatus comprising means for performing a method as claimed in any of claims or examples 11-20 or 57-66.

Example 71 includes a computing device arranged to implement or perform a method as claimed in any of claims or examples 11-20 or 57-66.

Example 72 includes a communications device arranged to implement or perform a method as claimed in any of claims or examples 11-20 or 57-66.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

What is claimed is:

1. An apparatus comprising:
detection logic to detect air exhaled by a user into a first computing device, wherein the air includes breath associated with the user;
sensing logic to sense the breath in the air;
sampling and evaluation logic to obtain a sample of the breath, and evaluate the sample;
messaging logic to generate a message based on the evaluation of the sample; and
communication/compatibility logic to present, via one or more output components, the message to the user via a user interface, wherein the message includes results of the evaluation of the breath sample.

2. The apparatus of claim 1, wherein the message comprises one or more of a brief overview of the user's health, a detailed analysis of the breath, a warning, an alert, a note, a reminder, and a conflict, wherein the message is presented in one or more forms including one or more of an audio message, a video message, an image message, a olfactory message, and a haptic message.

3. The apparatus of claim 1, wherein one or more portions of the air represent the breath including alveolar breath, wherein the sensing logic is further to sense the breath based on determination of one or more of concentration of carbon dioxide in the alveolar breath and relative humidity in the breath.

4. The apparatus of claim 1, further comprising identification/authentication logic to identify and authenticate at least one of the first computing device and the user.

5. The apparatus of claim 4, wherein the first computing device comprises a smart toothbrush.

6. The apparatus of claim 4, wherein the first computing device further comprises smart mobile computers including one or more of smartphones, tablet computers, head-mounted displays, head-mounted gaming displays, wearable glasses, wearable binoculars, smart jewelry, smartwatches, smartcards, and smart clothing items.

7. The apparatus of claim 1, further comprising energy harvesting logic to manage one or more power sources associated with the first computing device, wherein managing includes ensuring sufficient power supply to the first computing device from the one or more power sources, the one or more power sources having at least one of a rechargeable battery and a wireless charging plate.

8. The apparatus of claim 1, further comprising location and mapping logic to determine, in real-time, one or more locations associated with the first computing device, wherein the location and mapping logic is further to communicate, in real-time, the one or more locations to a second computing device, wherein the second computing device includes a server computer.

9. The apparatus of claim 8, wherein the location and mapping logic is further to continuously receive, via communication/compatibility logic, one or more notices relating to changing conditions associated with the one or more locations, wherein the one or more notices include a warning indicating an occurrence a dire condition associated with a location of the one or more locations.

10. The apparatus of claim 9, wherein the warning is further communicated to one or more computing devices associated with one or more medical personnel, wherein the one or more computing devices include one or more of desktop computers and mobile computers including one or more of smartphones, tablet computers, head-mounted displays, head-mounted gaming displays, wearable glasses, wearable binoculars, smart jewelry, smartwatches, smartcards, and smart clothing items.

11. A method comprising:
detecting air exhaled by a user into a first computing device, wherein the air includes breath associated with the user;
sensing the breath in the air;
obtaining a sample of the breath, and evaluating the sample;
generating a message based on the evaluation of the sample; and
presenting, via one or more output components, the message to the user via a user interface, wherein the message includes results of the evaluation of the breath sample.

12. The method of claim 11, wherein the message comprises one or more of a brief overview of the user's health, a detailed analysis of the breath, a warning, an alert, a note, a reminder, and a conflict, wherein the message is presented in one or more forms including one or more of an audio message, a video message, an image message, a olfactory message, and a haptic message.

13. The method of claim 11, wherein one or more portions of the air represent the breath including alveolar breath, wherein the sensing logic is further to sense the breath based on determination of one or more of concentration of carbon dioxide in the alveolar breath and relative humidity in the breath.

14. The method of claim 11, further comprising identifying and authenticating at least one of the first computing device and the user.

15. The method of claim 14, wherein the first computing device comprises a smart toothbrush.

16. The method of claim 14, wherein the first computing device further comprises smart mobile computers including one or more of smartphones, tablet computers, head-mounted displays, head-mounted gaming displays, wearable glasses, wearable binoculars, smart jewelry, smartwatches, smartcards, and smart clothing items.

17. The method of claim 11, further comprising managing one or more power sources associated with the first computing device, wherein managing includes ensuring sufficient power supply to the first computing device from the one or more power sources, the one or more power sources having at least one of a rechargeable battery and a wireless charging plate.

18. The method of claim 11, further comprising:
determining, in real-time, one or more locations associated with the first computing device; and
communicating, in real-time, the one or more locations to a second computing device, wherein the second computing device includes a server computer.

19. The method of claim 18, further comprising continuously receiving one or more notices relating to changing conditions associated with the one or more locations, wherein the one or more notices include a warning indicating an occurrence a dire condition associated with a location of the one or more locations.

20. The method of claim 19, wherein the warning is further communicated to one or more computing devices associated with one or more medical personnel, wherein the one or more computing devices include one or more of desktop computers and mobile computers including one or more of smartphones, tablet computers, head-mounted displays, head-mounted gaming displays, wearable glasses, wearable binoculars, smart jewelry, smartwatches, smartcards, and smart clothing items.

21. At least one machine-readable medium comprising a plurality of instructions, executed on a computing device, to facilitate the computing device to perform one or more operations comprising:
detecting air exhaled by a user into a first computing device, wherein the air includes breath associated with the user;
sensing the breath in the air;
obtaining a sample of the breath, and evaluating the sample;
generating a message based on the evaluation of the sample; and
presenting, via one or more output components, the message to the user via a user interface, wherein the message includes results of the evaluation of the breath sample.

22. The machine-readable medium of claim 21, wherein the message comprises one or more of a brief overview of the user's health, a detailed analysis of the breath, a warning, an alert, a note, a reminder, and a conflict, wherein the message is presented in one or more forms including one or more of an audio message, a video message, an image message, a olfactory message, and a haptic message.

23. The machine-readable medium of claim 21, wherein one or more portions of the air represent the breath including alveolar breath, wherein the sensing logic is further to sense the breath based on determination of one or more of concentration of carbon dioxide in the alveolar breath and relative humidity in the breath.

24. The machine-readable medium of claim 21, wherein the one or more operations further comprise identifying and authenticating at least one of the first computing device and the user.

25. The machine-readable medium of claim 24, wherein the first computing device comprises a smart toothbrush.

26. The machine-readable medium of claim 24, wherein the first computing device further comprises smart mobile computers including one or more of smartphones, tablet computers, head-mounted displays, head-mounted gaming displays, wearable glasses, wearable binoculars, smart jewelry, smartwatches, smartcards, and smart clothing items.

27. The machine-readable medium of claim 21, wherein the one or more operations further comprise managing one or more power sources associated with the first computing device, wherein managing includes ensuring sufficient power supply to the first computing device from the one or more power sources, the one or more power sources having at least one of a rechargeable battery and a wireless charging plate.

28. The machine-readable medium of claim 21, wherein the one or more operations further comprise:
determining, in real-time, one or more locations associated with the first computing device; and
communicating, in real-time, the one or more locations to a second computing device, wherein the second computing device includes a server computer.

29. The machine-readable medium of claim 28, wherein the one or more operations further comprise continuously receiving one or more notices relating to changing conditions associated with the one or more locations, wherein the one or more notices include a warning indicating an occurrence a dire condition associated with a location of the one or more locations.

30. The machine-readable medium of claim 29, wherein the warning is further communicated to one or more computing devices associated with one or more medical personnel, wherein the one or more computing devices include one or more of desktop computers and mobile computers including one or more of smartphones, tablet computers, head-mounted displays, head-mounted gaming displays, wearable glasses, wearable binoculars, smart jewelry, smartwatches, smartcards, and smart clothing items.
